(19) United States
(12) Patent Application Publication
(10) Pub. No.: US 2011/0054227 A1
(43) Pub. Date: Mar. 3, 2011

(54) Process to Protect Hydrogenation and Isomerization Catalysts Using a Guard Bed

(75) Inventors: Tin-Tack Peter Cheung, Kingwood, TX (US); Marvin M. Johnson, Bartlesville, OK (US); Darin B. Tiedtke, Kingwood, TX (US)

(73) Assignee: CHEVRON PHILLIPS CHEMICAL COMPANY LP, The Woodlands, TX (US)

(21) Appl. No.: 12/869,156

(22) Filed: Aug. 26, 2010

Related U.S. Application Data
(60) Provisional application No. 61/236,930, filed on Aug. 26, 2009.

(51) Int. Cl. C07C 11/06; C07C 5/05; C07C 11/04

(52) U.S. Cl. 585/16; 585/254

(57) ABSTRACT

Processes and an apparatus for hydrogenating highly unsaturated hydrocarbons contained in an effluent stream to an unsaturated hydrocarbons or isomerizing the highly unsaturated hydrocarbons to other highly unsaturated hydrocarbons are provided. The effluent stream is contacted with a guard bed to remove at least a portion of impurities contained within the process stream and to isomerize at least a portion of the highly unsaturated hydrocarbons. In an aspect, the guard bed comprises a solid sulfur adsorption/isomerization catalyst composition. In an aspect, the effluent stream is contacted with a catalyst that comprises an inorganic support, palladium, and silver.

### CROSS REFERENCE TO RELATED APPLICATIONS

This application claims priority to and the benefit of U.S. Provisional Patent Application No. 61/236930, filed Aug. 26, 2009, which is hereby incorporated by reference in its entirety.

### FIELD OF THE INVENTION

This invention relates to processes for the production of unsaturated hydrocarbons, and more particularly to a guard bed containing basic components used to remove hydrogenation and isomerization catalyst poisons.

### BACKGROUND

Unsaturated hydrocarbon compounds are commonly produced by thermal (pyrolytic) cracking of saturated hydrocarbon streams. For example, a stream containing a saturated hydrocarbon such as, for example, ethane, propane, butane, pentane, naphtha, and the like and combinations thereof can be fed into a cracking furnace. Within the cracking furnace and subsequent scrubbing, cooling, compression, drying and fractionation processes, the saturated hydrocarbon is converted to an effluent stream containing unsaturated hydrocarbon compounds such as monoolefins, which are also referred to as alkenes. Suitable monoolefins can include, for example, ethylene, propylene, and the like. Such unsaturated hydrocarbons are an important class of chemicals that find a variety of industrial uses. For example, ethylene can be used as a monomer or comonomer for producing a polyolefin, such as polyethylene. Other uses of unsaturated hydrocarbons are well known to those skilled in the art.

The unsaturated hydrocarbons produced by a thermal cracking process generally contain an appreciable amount of less desirable highly unsaturated hydrocarbons, such as alkynes or diolefins (which are also referred to as alkadienes). For commercial purposes, it is desirable to selectively hydrogenate the highly unsaturated hydrocarbons to the unsaturated hydrocarbons, such as propylene, but not to the saturated hydrocarbons such as propane, in a hydrogenation reaction. As an alternative example, propylene produced by thermal cracking of propane can be contaminated with a highly unsaturated hydrocarbon such as propadiene. For commercial purposes, it is desirable to isomerize the propadiene to another highly unsaturated hydrocarbon, such as methyl acetylene, which can then be selectively hydrogenated to the unsaturated hydrocarbon, such as propylene.

Selective hydrogenation catalysts comprising palladium and an inorganic support, such as alumina, are known catalysts for the hydrogenation of highly unsaturated hydrocarbons. In the case of the selective hydrogenation of acetylene to ethylene, a palladium and silver catalyst supported on an inorganic support can be employed. Such catalysts are disclosed in U.S. Pat. Nos. 4,484,015, 5,489,565, 7,247,760, and 7,417,007 the disclosures of which are incorporated herein by reference. The operating temperature for this hydrogenation process is selected to maximize hydrogenation of a highly unsaturated hydrocarbon to its corresponding unsaturated hydrocarbon while limiting the hydrogenation of the unsaturated hydrocarbon to the saturated hydrocarbon. Thus, the highly unsaturated hydrocarbon is removed from the product stream by conversion to a desired product and the amount of the desired product that is hydrogenated to a saturated hydrocarbon is minimized.

Impurities that are present in a highly unsaturated hydrocarbon stream can poison and deactivate a palladium-containing selective hydrogenation catalyst. Such impurities can include sulfur impurities, such as H₂S, carbonyl sulfide (COS), carbon disulfide, mercaptans, organic sulfides, organic disulfides, thiophene, organic trisulfides, organic tetrasulfides, and combinations thereof. In some instances, carbon monoxide can temporarily poison or inactivate such a palladium-containing selective hydrogenation catalyst. It is also generally known by those skilled in the art that when a sulfur impurity is present during the hydrogenation of highly unsaturated hydrocarbons to unsaturated hydrocarbons, the sulfur impurity can poison and deactivate the selective hydrogenation catalyst. This is especially true in a depropanizer hydrogenation process because the feed stream from the depropanizer being sent to the acetylene removal unit (also referred to as ARU) of such depropanizer hydrogenation process typically contains low levels of a sulfur compound(s) with the possibility of transient spikes in the level of such sulfur compound(s). Thus, the development of processes for the hydrogenation of highly unsaturated hydrocarbons to unsaturated hydrocarbons in the presence of a sulfur impurity would be a significant contribution to the art and to the economy.

Hydrogenation of methylacetylene proceeds more readily than propadiene, thus the conversion of the propadiene can be increased if the propadiene is first isomerized to methylacetylene. In front-end depropanizer ARU service, palladium without promoters usually provides a good conversion of the propadiene to methylacetylene because the palladium catalyzes the hydroisomerization of the propadiene to methylacetylene. However, it is well known that a palladium selective hydrogenation catalyst without promoters is a poor selective hydrogenation catalyst as far as in converting acetylene to ethylene. The selectivity is improved by the addition of silver to the palladium but the silver also drastically reduces the hydroisomerization activity of the palladium, thus greatly reducing the conversion of the propadiene.

### SUMMARY

Described are processes and an apparatus for hydrogenating a highly unsaturated hydrocarbon in an effluent stream to an unsaturated hydrocarbon or to other highly unsaturated hydrocarbons. The effluent stream is prepared from the gas product of a cracking furnace by various subsequent scrubbing, cooling, compression, drying and fractionation processes known to the olefins production industry. The effluent stream is contacted with a guard bed to remove at least a portion of impurities contained within the effluent stream and to isomerize at least a portion of the highly unsaturated hydrocarbon to produce a treated hydrocarbon stream. The treated hydrocarbon stream is then hydrogenated to produce the unsaturated hydrocarbon or other highly unsaturated hydrocarbon. In an aspect, the guard bed comprises a solid sulfur adsorption/isomerization catalyst composition. In some embodiments, the effluent stream is contacted with a selective hydrogenation catalyst composition comprising an inorganic support, palladium, and silver in the presence of hydrogen.

### DETAILED DESCRIPTION

The term “highly unsaturated hydrocarbon” refers to a hydrocarbon molecule having a triple bond or two or more double bonds between carbon atoms in the molecule. Examples of highly unsaturated hydrocarbons include, but are not limited to, aromatic compounds such as benzene and naphthalene; alkynes such as acetylene, methylacetylene (also referred to as propyne), and butynes; diolefins such as propadiene, butadienes, pentadienes (including isoprene), hexadienes, octadienes, and decadienes; and the like and combinations thereof.

The term “unsaturated hydrocarbon” refers to a hydrocarbon having no triple bonds, but having one or more double bonds; or a hydrocarbon in which the number of double bonds is one less, or at least one less, than that in the highly unsaturated hydrocarbon. Examples of unsaturated hydrocarbons include, but are not limited to, monoolefins such as ethylene, propylene, butenes, pentenes, hexenes, octenes, decenes, and the like and combinations thereof.

The term “hydrogenation” refers to a process that converts a highly unsaturated hydrocarbon to an unsaturated hydrocarbon or a saturated hydrocarbon such as an alkane. The term “selective” refers to such hydrogenation process in which a highly unsaturated hydrocarbon is converted to the unsaturated hydrocarbon without further hydrogenating the unsaturated hydrocarbon to the saturated hydrocarbon. Thus, for example, when the highly unsaturated hydrocarbon is converted to the unsaturated hydrocarbon without further hydrogenating such unsaturated hydrocarbon to a saturated hydrocarbon, the hydrogenation process is “more selective” than when such highly unsaturated hydrocarbon is hydrogenated to the unsaturated hydrocarbon and then further hydrogenated to a saturated hydrocarbon.

The term “isomerization” refers to a process that isomerizes the highly unsaturated hydrocarbon, such as a diolefin, to another highly unsaturated hydrocarbon, such as an alkyne, which, if desired, can be selectively hydrogenated to the unsaturated hydrocarbon such as a monoolefin in a subsequent hydrogenation process.

The term “guard bed” means any type of bed of particles within a vessel in which a solid sulfur adsorption/isomerization catalyst composition or selective hydrogenation catalyst composition, or both can be held and through which a fluid can be passed so that the fluid comes into contact with the solid sulfur absorption/isomerization catalyst composition, the selective hydrogenation catalyst composition, or both. Guard beds can be in any suitable vessel including, but not limited to, fixed bed reactors, packed towers, and the like. The guard bed can be in any suitable arrangement, such as, for example, a single bed, a mixed bed, a graded bed, or two separate beds. In a mixed bed, the solid sulfur adsorption/isomerization catalyst composition and selective hydrogenation catalyst can be uniformly mixed. In a graded bed, the guard bed can transition from the solid sulfur adsorption/isomerization catalyst composition to the selective hydrogenation catalyst by any suitable gradient. When in two separate beds, the solid sulfur adsorption/isomerization catalyst composition and the selective hydrogenation catalyst can be contained within a common vessel, or one or more vessels in series. Other suitable types of guard beds will be apparent to those of ordinary skill in the art and are to be considered within the scope of the present invention. As used herein, the term “fluid” denotes gas, liquid, vapor, or combinations thereof.

The term “raw gas stream” means a hydrocarbon stream containing unsaturated hydrocarbons and highly unsaturated hydrocarbons having a thermal cracking furnace as its origin. The term “effluent stream” means a hydrocarbon stream containing unsaturated hydrocarbons and highly unsaturated hydrocarbons produced from a raw gas stream from a cracking furnace having undergone one or more purification steps necessary to remove most of the moisture and light gasses. These processes can include, but are not limited to, any number of cooling, scrubbing, compression, drying, fractionation, or similar steps necessary to removing most of the moisture and light gasses. These processes are generally well known to one skilled in the art of producing unsaturated hydrocarbons.

In an embodiment, a process for hydrogenation of unsaturated hydrocarbons by first use of a solid sulfur adsorption/isomerization catalyst composition in a guard bed placed before the selective hydrogenation reactor. The solid sulfur adsorption/isomerization catalyst composition improves the subsequent hydrogenation process by isomerizing all or part of a highly unsaturated hydrocarbon (generally, a diolefin) to another highly unsaturated hydrocarbon (generally, an alkyne) and by removing all or part of hydrogenation catalyst poisons, such as sulfur compounds. For example, in the case of a guard bed placed before a depropanizer ARU reactor, the solid sulfur adsorption/isomerization catalyst composition is utilized to isomerize all or part of the propadiene content in the ARU reactor feed into methyl acetylene and to remove ARU reactor catalyst poisons, such as sulfur impurities. The solid sulfur adsorption/isomerization catalyst composition generally comprises an isomerization catalyst composition that isomerizes one highly unsaturated hydrocarbon into another highly unsaturated hydrocarbon and a sulfur adsorbent.

In an embodiment, a selective hydrogenation process for hydrogenating a highly unsaturated hydrocarbon contained in an unsaturated hydrocarbon feedstream to an unsaturated hydrocarbon is provided. In this embodiment, the highly unsaturated hydrocarbon feedstream is contacted with a guard bed to form a treated hydrocarbon feedstream. The treated hydrocarbon feedstream is contacted with a hydrogenation catalyst composition in the presence of hydrogen to produce the unsaturated hydrocarbon.

In another embodiment, a process for the isomerization of the highly unsaturated hydrocarbons contained within the effluent stream to other highly unsaturated hydrocarbons is provided. The effluent stream is contacted with the guard bed to remove at least a portion of impurities contained within the effluent stream and to isomerize the highly unsaturated hydrocarbons to produce one or more isomerized highly unsaturated hydrocarbons. The guard bed includes a solid sulfur adsorption/isomerization catalyst composition.

In an aspect, the highly unsaturated hydrocarbon is contained within the effluent stream. In some embodiments, the highly unsaturated hydrocarbon comprises between about 0.01 ppm to about 50,000 ppm based on the weight of the effluent stream. In some embodiments, the highly unsaturated hydrocarbon comprises between about 0.10 ppm to about 10,000 ppm by weight, of the effluent stream. Other components contained within the unsaturated hydrocarbon feedstream are described herein.

In an aspect, the highly unsaturated hydrocarbon feedstream comprises a hydrocarbon selected from at least one highly unsaturated hydrocarbon including between 2 to 10 carbon atoms per molecule. In an aspect, the effluent stream contains propadiene. More than one highly unsaturated hydrocarbon can be present in the highly unsaturated hydrocarbon feedstream.

Solid sulfur adsorption/isomerization catalyst compositions useful for the processes described herein comprise a supported catalyst composition, comprising cerium, magnesium, and an inorganic support. In some embodiments the selective hydrogenation catalyst composition comprises an inorganic support, palladium, and silver. In another aspect, the selective hydrogenation catalyst composition further comprises at least one alkali metal.

In some embodiments the solid sulfur adsorption/isomerization catalyst composition include supported alkaline metal hydroxides, supported alkaline metal oxides, supported alkaline metal carbonates, supported alkaline earth oxides, supported rare earth oxides, mixed oxides of alkaline earth metals, mixed oxides of rare earth metals, and combinations thereof supported on an inorganic support. Commercial adsorbents including, but not limited to, Selexsorb® COS adsorbents, which is sodium oxide on an alumina support, can also be used.

In some embodiments, the solid sulfur adsorption/isomerization catalyst composition functions principally as an isomerization catalyst. In this embodiment the solid sulfur adsorption/isomerization catalyst composition functions principally to isomerize the highly unsaturated hydrocarbon to the isomerized highly unsaturated hydrocarbon. This mode of operation can occur due to operational conditions, the amounts of impurities in the effluent stream, and the amounts and types of highly unsaturated hydrocarbons to be isomerized. In other embodiments, the solid sulfur adsorption/isomerization catalyst compositions can comprise supported cerium oxides, magnesium oxides, sodium oxides, and combinations thereof supported on an inorganic support.

Suitable inorganic support materials for the solid sulfur adsorption/isomerization catalyst compositions or hydrogenation catalyst compositions include materials such as alumina, silica, silicon carbide, amorphous and crystalline silica-aluminas, silica-magnesias, aluminophosphates, boria, titania, zirconia, clays, zeolitic materials and combinations thereof. In some embodiments, the inorganic support can be zeolitic materials having micro pores such as conventional zeolitic materials and molecular sieves can be used in the solid sulfur adsorption/isomerization catalyst composition.

The solid sulfur adsorption/isomerization catalyst composition can include several classes of materials known to be reactive toward typical sulfur contaminants or impurities, such as hydrogen sulfide, COS, mercaptans, and organic sulfides. Several metal oxides can be useful as sulfur adsorbents and can be employed as the bulk oxides or can be supported on an appropriate inorganic support material such as an alumina, silica, a zeolite, and combinations thereof. Representative metal oxides include those of the metals from Groups IA and IIA. Representative elements include Li, Na, K, Rb, Cs, Mg, Ca, Sr, Ba, and the like. One or more such metal oxides can be used in the solid sulfur adsorption/isomerization catalyst composition. Other suitable metal oxides will be apparent to those of ordinary skill in the art and are to be considered within the scope of the present invention. Compounds of the Group IA and IIA metals capable of functioning as sulfur adsorbents include, in addition to the oxides, the hydroxides, and carbonates.

Other metal oxides can be included in the solid sulfur adsorption/isomerization catalyst composition and include oxides of rare earth metals, namely those having atomic numbers between 57 and 71 in the lanthanide series. Representative rare earth metals include Ce, Pr, Nd, Pm, Sm, Eu, Gd, Tb, Dy, Mo, Er, Tm, Yb, Lu, and combinations thereof mixed with oxides of alkaline earth metals.

In some embodiments, regenerable sulfur adsorbents can be included in the solid sulfur adsorption/isomerization catalyst composition. Sulfur adsorbents that bind sulfur through physical adsorption can typically be regenerated through changes in the process temperature, pressure, and/or gas rate. Representative of such sulfur adsorbents are zeolitic materials, spinels, meso-, and microporous transition metal oxides. Other suitable sulfur adsorbents will be apparent to those of ordinary skill in the art and are to be considered within the scope of the present invention.

The solid sulfur adsorption/isomerization catalyst composition can be utilized in various bed configurations within the reactor. The choice of configuration may or may not be critical depending upon the objectives of the overall process, particularly when the processes described herein are integrated with one or more subsequent processes, or when the objective of the overall process is to favor the selectivity of one aspect of product quality relative to another. Various bed configurations are disclosed with the understanding that the selection of a specific configuration is tied to these other process objectives. In an embodiment, a bed configuration utilizing a common reactor where the sulfur adsorbent composition is placed upstream of the isomerization catalyst composition catalyst zone. One bed configuration consists of a stacked bed wherein the sulfur adsorbent composition catalyst is stacked, or layered, above and upstream of the isomerization catalyst composition. Stacked beds can either occupy a common reactor, or the sulfur adsorbent composition can occupy a separate reactor upstream of the reactor containing the isomerization catalyst composition. The separate reactor sequence can be used when it is desirable to operate the isomerization catalyst composition and the sulfur adsorbent composition at substantially different reactor temperatures or to facilitate frequent or continuous replacement of the sulfur adsorbent material.

The sulfur adsorbent zone can also contain a mixed bed wherein particles of a solid sulfur adsorption composition are intimately intermixed with those of an isomerization catalyst composition. In both the stacked and mixed bed configurations, the two components can share similar or identical shapes and sizes, or the particles of one can differ in shape and/or size from the particles of the second component. Because a simple physical separation of the bed components upon discharge or reworking can easily separate the solid sulfur adsorption composition from the isomerization catalyst composition, having different shapes and sizes can be beneficial.

In some embodiments, the solid sulfur adsorption/isomerization catalyst composition comprises (a) palladium such as palladium metal, palladium oxide, or combinations thereof; (b) optionally, silver and/or an alkali metal compound; and (c) support comprising cerium oxide, magnesium oxide, and an inorganic support. The palladium can be present as “skin” on or near the surface of the support and the silver and/or alkali metal compound can be distributed as skin with the palladium or throughout the solid sulfur adsorption/isomerization catalyst composition.

Generally, the cerium component of the solid sulfur adsorption/isomerization catalyst composition can be present in any weight percent that is effective isomerizing a highly unsaturated hydrocarbon (such as a diolefin) to another highly unsaturated hydrocarbon (such as an alkyne). Generally, the solid sulfur adsorption/isomerization catalyst composition contains cerium in the range of from about 0.01 weight percent cerium based on the total weight of the solid sulfur adsorption/isomerization catalyst composition to about 15 weight percent cerium; alternatively, in the range of from about 0.1 weight percent cerium to about 10 weight percent cerium; and alternatively, in the range of from 0.05 weight percent cerium to 5 weight percent cerium.

Generally, magnesium can be present in the solid sulfur adsorption/isomerization catalyst composition in any weight percent that is effective in isomerizing a highly unsaturated hydrocarbon (such as a diolefin) to another highly unsaturated hydrocarbon (such as an alkyne). Generally, the solid sulfur adsorption/isomerization catalyst composition comprises magnesium in the range of from about 0.01 weight percent magnesium based on the total weight of the solid sulfur adsorption/isomerization catalyst composition to about 15 weight percent magnesium; alternatively, in the range of from about 0.1 weight percent magnesium to about 10 weight percent magnesium; and alternatively, in the range of from 0.5 weight percent magnesium to 5 weight percent magnesium.

Generally, the molar ratio of magnesium to cerium (Mg:Ce molar ratio) in the solid sulfur adsorption/isomerization catalyst composition can be in the range of from about 0.01:1 to about 20:1; alternatively, in the range of from about 0.01:1 to about 15:1; and alternatively, in the range of from 0.01:1 to 10:1.

The solid sulfur adsorption/isomerization catalyst composition can comprise an inorganic support selected from the group consisting of alumina, titania, zirconia, and the like and combinations thereof. In some embodiments, the inorganic support is alumina. Generally, the alumina used in the solid sulfur adsorption/isomerization catalyst composition can be any suitable alumina such as, but not limited to, alpha alumina, beta alumina, delta alumina, eta alumina, gamma alumina, and the like and combinations thereof. In particular embodiments, the alumina is delta alumina. The alumina can also contain minor amounts of other ingredients, such as, for example, silica in a range of from about 1 weight percent silica to about 10 weight percent silica. Generally, in some embodiments, it is desirable to have a substantially pure alumina as a starting material for preparation of the solid sulfur adsorption/isomerization catalyst composition. In some embodiments, the starting material is substantially pure delta alumina. The alumina used as the starting material for preparation of the solid sulfur adsorption/isomerization catalyst composition can be made by any manner or method(s) known in the art. As an example, a suitable commercially available alumina useful in preparing the catalyst composition according to the inventive processes described herein are delta alumina tablets or extrudate pellets or spheres, such as those manufactured by Süd-Chemie, Inc., Louisville, Ky. In some embodiments, the commercially available alumina is delta alumina tablets.

In lieu of or in addition to a silver component of the selective hydrogenation catalyst composition, an alkali metal compound can be used. Any alkali metal-containing compound can be used in the catalyst composition as long as the resulting selective hydrogenation catalyst composition is effective in selectively hydrogenating a highly unsaturated hydrocarbon to the unsaturated hydrocarbon. Suitable examples of alkali metal compounds for use in incorporating, such as by impregnating, the alkali metal compound into, onto, or with the inorganic support generally include, but are not limited to, alkali metal halides, alkali metal hydroxides, alkali metal carbonates, alkali metal bicarbonates, alkali metal nitrates, alkali metal carboxylates, and the like and combinations thereof. In an aspect, the alkali metal compound is an alkali metal halide; and alternatively, the alkali metal compound is an alkali metal iodide or an alkali metal fluoride. Generally, the alkali metal of such alkali metal compound is selected from the group consisting of potassium, rubidium, cesium, and the like and combinations thereof. Alternatively, the alkali metal of such alkali metal compound is potassium. Alternatively, the alkali metal compound is potassium iodide (KI); and alternatively, the alkali metal compound is potassium fluoride (KF).

Further examples of suitable alkali metal compounds include sodium fluoride, potassium fluoride, lithium fluoride, rubidium fluoride, cesium fluoride, sodium iodide, potassium iodide, lithium iodide, rubidium iodide, cesium iodide, sodium chloride, potassium chloride, lithium chloride, rubidium chloride, cesium chloride, sodium bromide, potassium bromide, lithium bromide, rubidium bromide, cesium bromide, sodium hydroxide, potassium hydroxide, lithium hydroxide, rubidium hydroxide, cesium hydroxide, sodium oxide, potassium oxide, lithium oxide, rubidium oxide, cesium oxide, sodium carbonate, potassium carbonate, lithium carbonate, rubidium carbonate, cesium carbonate, sodium nitrate, potassium nitrate, lithium nitrate, rubidium nitrate, cesium nitrate, and the like and combinations thereof.

Generally, the selective hydrogenation catalyst composition comprises an alkali metal compound in the range of from about 0.001 weight percent alkali metal compound to about 10 weight percent alkali metal compound based on the total weight of the selective hydrogenation catalyst composition. Alternatively, the selective hydrogenation catalyst composition comprises an alkali metal compound in the range of from about 0.005 weight percent alkali metal compound to about 5 weight percent alkali metal; and alternatively, in the range of from about 0.01 weight percent alkali metal compound to about 2 weight percent alkali metal. Generally, the weight ratio of an alkali metal compound to palladium in the selective hydrogenation catalyst is in the range of from about 0.05:1 to about 500:1. Alternatively, the weight ratio of an alkali metal compound to palladium is in the range of from about 0.1:1 to about 200:1; and alternatively, in the range of from about 0.2:1 to about 100:1. In some embodiments, the selective hydrogenation catalyst comprises about 0.01 to about 1 weight % palladium.

According to some embodiments, an isomerization process is provided. The isomerization process comprises contacting a hydrocarbon-containing fluid that comprises one or more highly unsaturated hydrocarbons with the solid sulfur adsorption/isomerization catalyst composition, in the presence of hydrogen in a guard bed under an isomerization condition to isomerize the one or more highly unsaturated hydrocarbons to another highly unsaturated hydrocarbon. In some embodiments, the solid sulfur adsorption/isomerization catalyst composition comprises cerium, magnesium, and an inorganic support as disclosed herein. The guard bed precedes the selective hydrogenation process in which the highly unsaturated hydrocarbons are hydrogenated to form unsaturated hydrocarbons. In some aspects, the unsaturated hydrocarbons can be further hydrogenated to form the saturated hydrocarbons.

In some embodiments, the highly unsaturated hydrocarbon is selected from the group consisting of alkynes, conjugated dienes, cumulative dienes, and combinations thereof. Examples of suitable alkynes include, but are not limited to, acetylene, methyl acetylene, 1-butyne, 2-butyne, 1-pentyne, 2-pentyne, 3-methyl-1-butyne, 1-hexyne, 1-heptyne, 1-octyne, 1-nonyne, 1-decyne, and the like and combinations thereof. In an embodiment, the alkynes are selected from the group consisting of acetylene, methyl acetylene, and combinations thereof. Other suitable highly unsaturated hydrocarbons will be apparent to those of skill in the art and are to be considered within the scope of the present invention.

Examples of suitable diolefins include those containing in the range of from 3 carbon atoms per molecule to about 12 carbon atoms per molecule. Such diolefins include, but are not limited to, propadiene, 1,2-butadiene, 1,3-butadiene, isoprene, 1,2-pentadiene, 1,3-pentadiene, 1,4-pentadiene, 1,2-hexadiene, 1,3-hexadiene, 1,4-hexadiene, 1,5-hexadiene, 2-methyl-1,2-pentadiene, 2,3-dimethyl-1,3-butadiene, heptadienes, methylhexadienes, octadienes, methylheptadienes, dimethylhexadienes, ethylhexadienes, trimethylpentadienes, methyloctadienes, dimethylheptadienes, ethyloctadienes, trimethylhexadienes, nonadienes, decadienes, undecadienes, dodecadienes, cyclopentadienes, cyclohexadienes, methylcyclopentadienes, cycloheptadienes, methylcyclohexadienes, dimethylcyclopentadienes, ethylcyclopentadienes, dicyclopentadiene, and the like and combinations thereof. In some embodiments, the diolefins are propadiene, 1,2-butadiene, 1,3-butadiene, pentadienes (such as 1,3-pentadiene, 1,4-pentadiene, isoprene), cyclopentadienes (such as 1,3-cyclopentadiene), dicyclopentadiene (also known as tricyclo[5.2.1]2,6-deca-3,8-diene), and combinations thereof. Other suitable diolefins will be apparent to those of skill in the art and are to be considered within the scope of the present invention.

In some embodiments, the highly unsaturated hydrocarbon can be a conjugated diene selected from the group consisting of 1,3-butadiene, isoprene, pentadienes, hexadienes, heptadienes, octadienes, cyclopentadienes, dicyclopentadiene, methylhexadienes, methylheptadienes, dimethylhexadienes, ethylhexadienes, trimethylpentadienes, methyloctadienes, dimethylheptadienes, ethyloctadienes, trimethylhexadienes, nonadienes, decadienes, undecadienes, dodecadienes, cyclohexadienes, methylcyclopentadienes, cycloheptadienes, methylcyclohexadienes, dimethylcyclopentadienes, ethylcyclopentadienes, and combinations thereof.

In some embodiments, the highly unsaturated hydrocarbon is a cumulative diene selected from the group consisting of propadiene, 1,2-butadiene, pentadienes, hexadienes, heptadienes, octadienes, methylhexadienes, methylheptadienes, dimethylhexadienes, ethylhexadienes, trimethylpentadienes, methyloctadienes, dimethylheptadienes, ethyloctadienes, trimethylhexadienes, nonadienes, decadienes, undecadienes, dodecadienes, and combinations thereof.

In an embodiment, the diolefins disclosed herein are isomerized to their corresponding alkyne(s) containing the same number of carbon atoms per molecule as the diolefins. In some embodiments, propadiene is isomerized to methyl acetylene; 1,2-butadiene is isomerized to 1-butyne and 2-butyne; 1,3-pentadiene and 1,4-pentadiene are isomerized to 1-pentyne, 2-pentyne, and 3-methyl-1-butyne; 1-hexadiene is isomerized to 1-hexyne; and 1-heptadiene is isomerized to 1-heptyne. Other appropriate isomerization reactions will be apparent to those of ordinary skill in the art and are to be considered within the scope of the present invention.

The unsaturated hydrocarbon feedstream that is sent to the guard bed can contain an impurity at a level that does not significantly interfere with the hydrogenation process of the highly unsaturated hydrocarbon to the unsaturated hydrocarbon as disclosed herein and/or an isomerization process of the highly unsaturated hydrocarbon to another highly unsaturated hydrocarbon as disclosed herein. The term “impurity” as used herein denotes any component in a hydrocarbon-containing stream that is not a major component and does not materially affect any of the reactions described herein. Examples of impurities other than highly unsaturated hydrocarbons, such as, for example, an alkyne or a diolefin include, but are not limited to hydrogen sulfide, carbonyl sulfide (COS), carbon disulfide (CS₂), mercaptans (RSH), organic sulfides (RSR), organic disulfides (RSSR), thiophenes, organic trisulfides, organic tetrasulfides, and the like and combinations thereof. In the examples, each R can be the same or different and can be an alkyl or cycloalkyl or aryl group containing from about 1 carbon atom to about 15 carbon atoms. In some embodiments, each R can be an alkyl or cycloalkyl or aryl group containing from 1 carbon atom to about 10 carbon atoms. It is within the scope of the present disclosure to have additional compounds (such as carbon monoxide, water, alcohols, ethers, aldehydes, ketones, carboxylic acids, esters, other oxygenated compounds, and the like and combinations thereof) present in the guard bed feed, as long as they do not significantly interfere with the hydrogenation process of the highly unsaturated hydrocarbon to the unsaturated hydrocarbon as disclosed herein and/or an isomerization process of the highly unsaturated hydrocarbon to another highly unsaturated hydrocarbon as disclosed herein.

The isomerization and sulfur removal processes can be conducted separately or simultaneously in separate zones or in the same zone within the guard bed.

Generally, hydrogen is fed into the guard bed in an amount in the range of from about 0.1 mole of hydrogen employed for each mole of highly unsaturated hydrocarbon present in the guard bed feed to about 1000 moles of hydrogen employed for each mole of highly unsaturated hydrocarbon present in the guard bed feed. In some embodiments, hydrogen gas can be fed into the guard bed. In an embodiment, an isomerization process comprises the presence of hydrogen in an amount in the range of from about 0.5 mole to about 500 moles of hydrogen employed for each mole of highly unsaturated hydrocarbon; and alternatively, in the range of from about 0.7 mole to about 200 moles of hydrogen employed for each mole of highly unsaturated hydrocarbon. In some embodiments, the isomerization process includes the presence of hydrogen gas.

Generally, the isomerization zone of the guard bed is maintained at a temperature in the range of from about 10° C. to about 300° C.; alternatively, in the range of from about 20° C. to about 250° C.; and alternatively, in the range of from about 20° C. to about 200° C. A suitable pressure is generally in the range of from about 15 pounds per square inch gauge (psig) to about 2000 psig; alternatively, in the range of from about 50 psig to about 1500 psig; and alternatively, in the range of from about 100 psig to about 1000 psig.

The processes of this invention can be utilized as a stand-alone process for purposes of various fuels, lubes, and chemical applications. Alternatively, the processes can be combined and integrated with other processes in a manner so that the net process affords product and process advantages and improvements relative to the individual processes not combined.

In some embodiments, the processes described herein can be operated as a batch process. In some embodiments, the processes described herein can be operated as a continuous process.

In addition to the processes described herein, an apparatus is described that comprises a cracking furnace, a guard bed and a selective hydrogenation catalyst bed. In some embodiments the cracking furnace can be a thermal cracking furnace. In other embodiments the guard bed containing the solid sulfur adsorption/isomerization catalyst composition and the selective hydrogenation catalyst composition are contained within a mixed bed. In an Embodiment the mixed bed may be a graded bed. The graded bed can be used in a manner where the graded bed can transition from the solid sulfur adsorption/isomerization catalyst composition to the selective hydrogenation catalyst composition. The guard bed can include the solid sulfur adsorption/isomerization catalyst composition. The catalyst bed can also include the selective hydrogenation catalyst comprising an inorganic support, palladium, and silver. In an embodiment the guard bed containing the solid sulfur adsorption/isomerization catalyst composition has the solid sulfur adsorption/isomerization catalyst composition in a separate bed from the selective hydrogenation catalyst composition. These two separate catalyst beds may be arranged in a number of configurations. In an aspect, the guard bed and the selective hydrogenation catalyst bed can be contained within a common vessel. In an aspect, the guard bed is within one or more parallel vessels and the selective hydrogenation catalyst bed is contained within one or more parallel vessels. In other aspects, the solid sulfur adsorption/isomerization catalyst composition guard is within one or more parallel vessels and the selective hydrogenation catalyst bed is contained within one or more parallel vessels. In some embodiments, a heat exchanger can be located between the guard bed and the selective hydrogenation catalyst bed. In other words, the heat exchanger can be downstream of the guard bed and upstream of the selective hydrogenation catalyst bed.

The invention has been described with reference to certain preferred embodiments. However, as obvious variations thereon will become apparent to those of skill in the art, the invention is not to be limited thereto.

### EXAMPLES

The invention is further illustrated by the following examples, which are not to be construed in any way as imposing limitations to the scope of this invention. Various other aspects, embodiments, modifications, and equivalents thereof which, after reading the description herein, may suggest themselves to one of ordinary skill in the art without departing from the spirit of the present invention or the scope of the appended claims.

### Example 1

### Preparation of a Solid Sulfur Adsorption/Isomerization Catalyst Composition

200 g of distilled water was added to 57.4 g of KOH pellets and swirled until the KOH pellets dissolved to form a KOH/water solution. The KOH/water solution was added slowly to 343 g of Al₂O₃ (oil drop spheres) at ambient temperature while the Al₂O₃ was stirred. Upon completion of the addition of the KOH/water solution, the resultant material was dried in air in a 120° F. drying oven for 3 hours. After 3 hours in the drying oven, the KOH/Al₂O₃ was heated at the temperatures, times, and purge conditions listed in Table 1.

TABLE 1

| Temperature | Time | Purge Gas |
|---|---|---|
| 120° C. | 2 hr. | Air |
| 230° C. | 2 hr. | Air |
| 535° C. | 16 hr. | Air |
| 535° C. | 3 hr. | N₂ |

The KOH/Al₂O₃ was allowed to cool under flowing nitrogen to produce the residual KOH/Al₂O₃ product, Sample A, which was analyzed with the results being shown in Table 2.

TABLE 2

| Element | Wt. % |
|---|---|
| Al | 40.90 |
| K | 7.20 |
| Na | 0.08 |
| Mg | 0.07 |

### Example 2

### Isomerization Screening of Sample A

20 ml of Sample A (from Example 1) was placed in a ½″ I.D. stainless steel reactor that was heated to 400° F. with 100 cc/min flow of H₂ under atmospheric pressure. The reactor was then allowed to cool to 165° F. over a period of about fifteen hours. The reactor was maintained under H₂ flow during cooling. Sample A was then fed into the reactor at 700 cc/min along with H₂ at 200 cc/min at a pressure of 200 psig. Reactor effluent was examined at three different temperatures, as shown in Table 3.

TABLE 3

| | Feed | #1 | #2 | #3 |
|---|---|---|---|---|
| Temp (° F.) | | 165 | 107 | 203 |
| wt % C₆+ | 0.01 | 0.00 | 0.00 | 0.00 |
| wt % Methane | 20.22 | 20.15 | 20.16 | 20.12 |
| wt % ethane + ethylene | 56.03 | 56.47 | 56.31 | 56.41 |
| wt % propane + Acetylene | 0.33 | 0.33 | 0.34 | 0.34 |
| wt % Propylene | 22.61 | 22.24 | 22.36 | 22.30 |
| wt % Propadiene | 0.37 | 0.10 | 0.19 | 0.10 |
| wt % Methylacetylene | 0.40 | 0.67 | 0.60 | 0.68 |
| wt % Butane | 0.00 | 0.00 | 0.04 | 0.00 |
| wt % propadiene + methylacetylene | 0.77 | 0.77 | 0.79 | 0.79 |
| % propadiene conversion | — | 73.64 | 47.83 | 71.74 |
| % methylacetylene conversion | — | −67.16 | −48.26 | −70.15 |
| wt % ethane make | — | −0.044 | −0.038 | 0.001 |

Note:
wt % is on the basis of the hydrocarbon stream analyzed (i.e. feed or reactor effluent).

At all temperatures significant percentages of the methylacetylene was converted to other products as evidenced by the negative conversion shown in Table 3. At temperatures 1 and 2 small amounts of ethane were converted to other products, while at temperature 3 a small amount of net ethane was produced. While not wanting to be limited by theory the production of ethane at this higher temperature is due in some part to the over hydrogenation the highly unsaturated hydrocarbon.

We claim:

1. A selective hydrogenation process for hydrogenating a highly unsaturated hydrocarbon in an effluent stream to an unsaturated hydrocarbon comprising the steps of:
contacting the effluent stream with a guard bed to remove at least a portion of impurities contained within the effluent stream and to isomerize at least a portion of the highly unsaturated hydrocarbon thereby forming a treated hydrocarbon stream, wherein the guard bed comprises a solid sulfur adsorption/isomerization catalyst composition; and
hydrogenating the treated hydrocarbon stream in the presence of a selective hydrogenation catalyst composition comprising an inorganic support, palladium, and silver to produce the unsaturated hydrocarbon.

2. The process of claim 1, wherein the selective hydrogenation catalyst composition further comprises at least one alkali metal compound selected from the group consisting of alkali metal halides, alkali metal hydroxides, alkali metal carbonates, alkali metal bicarbonates, and combinations thereof.

3. The process of claim 1, wherein the solid sulfur adsorption/isomerization catalyst composition is selected from the group consisting alkaline metal hydroxides, alkaline metal oxides, alkaline metal carbonates, alkaline earth oxides, mixed oxides of alkaline earth metals, mixed oxides of rare earth metals, rare earth metal oxides, and combinations thereof supported on an inorganic support.

4. The process of claim 1, wherein the solid sulfur adsorption/isomerization catalyst composition is selected from the group consisting of cerium oxides, magnesium oxides, sodium oxides, and combinations thereof supported on an inorganic support.

5. The process of claim 1, wherein the highly unsaturated hydrocarbon is selected from the group of alkynes, conjugated dienes, cumulative dienes, and combinations thereof.

6. The process of claim 1, wherein the effluent stream comprises propadiene.

7. The process of claim 1 wherein at least a portion of the highly unsaturated hydrocarbon is isomerized to an isomerized highly unsaturated hydrocarbon.

8. The process of claim 1, wherein the impurities are selected from the group consisting of hydrogen sulfide, carbonyl sulfide, carbon disulfide, mercaptans, organic sulfides, organic disulfides, thiophene, organic trisulfides, organic tetrasulfides, and combinations thereof.

9. The process of claim 1, wherein the solid sulfur adsorption/isomerization catalyst composition functions principally to isomerize the highly unsaturated hydrocarbon to an isomerized highly unsaturated hydrocarbon.

10. The process of claim 3, wherein the inorganic support is selected from the group consisting of titania, zirconia, alpha alumina, beta alumina, delta alumina, eta alumina, gamma alumina, and combinations thereof.

11. The process of claim 1, wherein the solid sulfur adsorption/isomerization composition comprises an alkali metal compound selected from the group consisting of alkali metal halides, alkali metal hydroxides, alkali metal carbonates, alkali metal bicarbonates, and combinations thereof.

12. The process of claim 11, wherein the alkali metal compound is selected from the group consisting of sodium hydroxide, potassium hydroxide, lithium hydroxide, rubidium hydroxide, cesium hydroxide, sodium oxide, potassium oxide, lithium oxide, rubidium oxide, cesium oxide, sodium carbonate, potassium carbonate, lithium carbonate, rubidium carbonate, cesium carbonate, and combinations thereof.

13. The process of claim 1 wherein the solid sulfur adsorption/isomerization catalyst composition and a selective hydrogenation catalyst composition are both contained within the guard bed.

14. The process of claim 13 wherein the guard bed is a graded bed.

15. The process of claim 14 wherein the graded bed transitions from the solid sulfur adsorption/isomerization catalyst composition to the selective hydrogenation catalyst composition.

16. The process of claim 1 wherein the solid sulfur adsorption/isomerization catalyst composition and the selective hydrogenation catalyst composition are contained within one or more vessels in series.

17. The process of claim 16, wherein the solid sulfur adsorption/isomerization catalyst composition bed and the selective hydrogenation catalyst bed are contained within a common vessel.

18. The process of claim 16, wherein the solid sulfur adsorption/isomerization catalyst composition bed is within one or more parallel vessels and the selective hydrogenation catalyst bed is contained within one or more parallel vessels.

19. The process of claim 16, further comprising a heat exchanger located downstream of the solid sulfur adsorption/isomerization catalyst composition bed and upstream of the selective hydrogenation catalyst bed.

20. An ethylene stream produced by the process of claim 1.

21. A propylene stream produced by the process of claim 1.
